(19) United States
(12) Patent Application Publication
(10) Pub. No.: US 2011/0287947 A1
(43) Pub. Date: Nov. 24, 2011

(54) Tethered Conformation Capture

(75) Inventors: Lin Chen, Los Angeles, CA (US); Reza Kalhor, Los Angeles, CA (US)

(73) Assignee: UNIVERSITY OF SOUTHERN CALIFORNIA, Los Angeles, CA (US)

(21) Appl. No.: 12/782,616

(22) Filed: May 18, 2010

(51) Int. Cl. C40B 30/00; C12Q 1/68

(52) U.S. Cl. 506/7; 435/6.19

(57) ABSTRACT

Disclosed are methods and systems for determining the three-dimensional structure of chromatin in eukaryotic cells. More specifically, disclosed are methods and systems for obtaining chromatin structural information by surface immobilization, i.e tethering crosslinked protein:DNA complexes and/or ligated DNA complexes to media such as beads, gels, and or matrices during the conformation capture assay. In general, the method includes contacting a cell with a cross-linking reagent to cross-link DNA and protein in the cell; lysing the cell, producing cross-linked protein:DNA complexes by cutting the chromatin using a chemical, physical or enzymatic method, substantially immobilizing the cross-linked protein:DNA complexes, ligating the cross-linked protein:DNA complexes intramolecularly such that the ligated protein:DNA complexes represent structural organization of the chromatin; characterizing the ligated DNA by sequencing or other methods; and identifying any structural organization of the chromatin. The structural organization preferably includes information relating to interacting loci of the chromatin.

This invention was made with government support under Contract Nos. R01 GM064642, R01 HL076334 and R01 GM077320 awarded by the National Institute of Health. The government has certain rights in the invention.

### FIELD OF THE INVENTION

The present invention, Tethered Conformation Capture (TCC) relates in general to methods and systems for determining the three-dimensional structure of chromatin in Eukaryotic Cells. More specifically, the invention provides improved methods and systems for obtaining chromatin structural information by surface immobilization, i.e tethering crosslinked protein:DNA complexes and/or ligated DNA complexes to media such as beads, gels, matrices during the conformation capture assay.

### BACKGROUND OF THE INVENTION

The three-dimensional (3D) organization of eukaryotic genomes plays an important role in various nuclear processes such as transcription, replication, and DNA repair (Wolffe, 1998). Beyond packaging by nucleosomes, the folding and spatial arrangement of chromatin can regulate different nuclear processes (Cremer et al., 2006; Cremer and Cremer, 2001). Chromatin structure refers to the spatial organization and higher-order structure of DNA loci in the nucleus of cells. While involvement of the spatial organization of loci or conformation of the chromatin fiber has been shown in some examples (Osborne et al., 2004; Lee et al., 2005; Spilianakis and Flavell, 2004; Cai et al., 2007), understanding 3D organization of chromatin at a genome level remains elusive.

The ability to analyze 3D organization of chromatin has been hampered by technical limitations. Most of the methods that are used to address 3D organization of the genome have low resolution and throughput. High-throughput methods can enhance our ability to understand the spatial arrangement of loci. Methods that provide higher resolution, in addition to high throughput, can enable better evaluation of the conformation of the chromatin fiber.

The first developed conformation capture method, 3C, quantifies the interaction frequency of two loci by measuring the frequency of ligation between them in a locus specific polymerase chain reaction (PCR) (Dekker et al., 2002). The closely related method of 5C enables multiplexing 3C for several pairs of loci by ligation-mediated amplification of locus-specific probes (Dostie et al., 2006). In a different approach, 4C identifies various interaction partners of a chosen locus by amplifying all fragments ligated to that locus in an inverse PCR followed by hybridization to microarrays or sequencing (Simonis et al., 2006; Zhao et al., 2006). This first generation of capture methods is limited in scope in that it can only evaluate a few chosen interactions at a time. Therefore, none of these methods provides a genomic or regional context for the interaction frequencies to reveal the more significant aspects of the 3D organization of the locus or changes therein. Analysis of interaction frequencies out of genomic or regional context can be misleading about causal relationship between various aspects of chromatin organization and the function of the loci.

Recently a new method called Hi-C, combined the basic principle of conformation capture approaches with ultra-high throughput sequencing to detect interactions of chromatin genome-wide and unbiased to any locus (Lieberman-Aiden et al., 2009). Like the other conformation capture methods, Hi-C depends on lower concentrations to reduce intermolecular ligations.

Conformation capture techniques are the most commonly used methods to analyze the spatial arrangement of chromatin at a molecular level. These assays typically rely on intramolecular ligation of crosslinked DNA fragments and the elimination of intermolecular ligation of non-crosslinked DNA fragments to preserve the 3D organization of chromatin during the conformation capture technique. This is typically done by ligation at low concentration to minimize intermolecular ligation. These low concentrations are typically done by using very dilute solutions. Depending on the conformation capture method, the frequency of certain intramolecular ligation events can then measured by various techniques. Ligation frequency between two loci represents the frequency with which they interact.

The inventors have found, however, that ligation at low concentrations is disadvantageous because it extremely difficult to use the technique in conformation capture with both high resolution and low noise. The inventors have discovered that low concentrations are not effective at eliminating the ligation between different crosslinked DNA complexes. These intermolecular ligations, which manifest as false interactions in techniques such as Hi-C, appear to be a major source of noise in conformation capture assays.

Moreover, the theoretical resolution limit of a genome-wide conformation capture assay such as Hi-C is determined by the size of DNA fragments generated during the conformation capture method. Smaller DNA fragments result in higher resolution conformation capture, but also result in high concentration of DNA fragments which in turn result in increased intermolecular ligation reactions and increased noise. It is thus very difficult using known methods to achieve both high resolution and low noise in known conformation capture techniques.

### SUMMARY OF THE INVENTION

It is one object of the present invention to provide methods and systems for the determination of 3-dimensional chromatin structure at higher resolutions and with less noise.

It is another object of the present invention to provide methods and systems of genome-wide conformation capture which substantially eliminate intermolecular ligation during the conformation capture technique.

Another object of the present invention is to provide a new approach for high-throughput genome-wide analysis of 3D organization of chromatin having high resolution and low noise. This approach significantly reduces experimental noise by using surface immobilization rather than dilution for promoting intramolecular ligations. Surface immobilization makes possible more powerful analysis of global 3D arrangement of the genome and higher resolution evaluation of local chromatin conformation.

It is one discovery of the present invention that surface-immobilization of complexes, in contrast to reducing the concentration, effectively diminishes ligation between complexes. This renders conformation capture more effective by dramatically increasing the signal to noise ratio. Surface immobilization also enables more intricate modifications being carried out on cross-linked chromatin. Additionally, it paves the way for automation of such reactions.

One embodiment of the present invention is a method directed to a genome-wide conformation capture method of determining the three-dimensional arrangement of chromatin in a cell. The method comprises contacting a cell with a cross-linking reagent to cross-link DNA and protein in the cell such that the structural organization of the chromatin or other protein:DNA complexes is preserved; lysing the cell, producing cross-linked protein:DNA complexes by cutting the chromatin using a chemical, physical or enzymatic method, substantially immobilizing the cross-linked protein:DNA complexes, connecting the cross-linked protein:DNA complexes intramolecularly such that the connected protein:DNA complexes represent structural organization of the chromatin; characterizing the connected DNA by sequencing or other methods; and identifying any structural organization of the chromatin. Preferably, the sequencing is massively parallel or ultra-high throughput sequencing. The structural organization preferably includes information relating to interacting loci of the chromatin.

In a further preferred embodiment of the present invention, the protein:DNA complexes are cut by restriction digestion. Preferably, the chromatin is digested with a restriction enzyme that produces a 5′ overhang of at least two non-identical bases, and the 5′ overhang is subsequently blunted. In one embodiment, blunting may be done with nucleotide analogues, and more preferably, a biotinylated nucleotide and nuclease resistant nucleotide is used for blunting. Preferably, a 2-deoxy-nucleoside-5′-(alpha-thio)-triphosphate is used in blunting.

In connection with the preferred embodiment of the present invention, the protein:DNA complexes are substantially immobilized by tethering the protein:DNA complexes to one or more media. The media may be one or more media selected from the group consisting of beads, chips, colloids, matrices, and gels, and the protein:DNA complexes may be tethered on the surface of or on the inside of the media. In a preferred embodiment, the protein:DNA complexes are substantially immobilized by a covalent bond between the side-chains of the amino acids of the proteins of chromatin and a reactive chemical group on the surface or inside of one or more media selected from the group consisting of beads, chip, colloids, matrix, and gel.

In a further preferred embodiment of the present. invention, the protein:DNA complexes are substantially immobilized by modifying the proteins of the chromatin so to tether the modified protein:DNA complexes to the surface or inside of one or more media selected from the group consisting of beads, colloids, matrices, and gels. In connection with this embodiment, the protein:DNA complexes may be substantially immobilized by biotinylating the proteins of the chromatin so to tether the biotinylated protein:DNA complexes to the biotin binding surfaces such as streptavidin-coated surfaces selected from but not limited to the group consisting of chips or beads. Instead of streptavidin itself, variations such as avidin and neutravidin can be used. The thiol groups of the proteins, including the thiol groups of cysteine residues, may be biotinylated so as to anchor the biotinylated protein:DNA complexes to streptavidin, avidin or neutravidin coated surfaces.

Alternatively, the protein:DNA complexes may be substantially immobilized by biotinylating the N-termini and lysine residues of the proteins of the chromatin so to tether the biotinylated protein:DNA complexes to streptavidin coated chips or beads. Or the protein:DNA complexes may be substantially immobilized by biotinylating the glutamate or aspartate residues of the proteins of the chromatin so to anchor the biotinylated protein:DNA complexes to streptavidin or related coated chips or beads.

In a further embodiment of the present invention, thiol groups are added to the proteins of chromatin through a chemical reagent. The thiol groups may added to the proteins of chromatin through reacting the proteins of chromatin with an aminothiol and a crosslinking reagent. The thiol groups may be added to the lysines and N-termini of the proteins of chromatin by reacting them with a cross-linking reagent and cysteamine, for instance, by reacting them with formaldehyde and cysteamine.

Preferably, ends of cross-linked protein:DNA complexes are connected intramolecularly by ligation, and more preferably by blunt-ended ligation using DNA ligase. After the ligating step, protein:DNA complexes that have not undergone ligation are preferably removed.

Another embodiment of the present invention is directed to a tethered conformation capture method in which crosslinked protein-DNA complexes are immobilized on a surface where most reactions take place. According to this method, cells are crosslinked with formaldehyde and treated with Iodoacetyl-PEG₂-Biotin to biotinylate the cysteine residues of all proteins. Chromatin is then digested with a restriction enzyme that leaves 5′ overhangs. After digestion, crosslinked protein-DNA complexes are immobilized on the surface of streptavidin-coated magnetic beads through biotinylated proteins and excess streptavidin is blocked. 5′ overhangs are filled in with an α-thio-triphosphate containing nucleotide analogue inserted before a biotinylated nucleotide. Blunt DNA ends are then ligated while immobilization prevents free diffusion of the complexes and therefore promotes intramolecular ligations. After ligation, DNA is purified which separates it from the surface and crosslinked proteins. The biotinylated nucleotides on DNA ends that have not participated in ligation are then removed using E. coli Exonuclease III (ExoIII). ExoIII catalyzes removal of mononucleotides from 3′-hydroxyl termini of duplex DNA until it encounters the exonuclease resistant phosphorothioate bond, which is inserted on the 5′ of the biotinylated nucleotide by incorporation of the a-thio-triphosphate containing nucleotide. After exonuclease treatment, DNA is sheared and the fragments that include a ligation junction and have therefore maintained biotin, are isolated on streptavidin-coated magnetic beads. A library is prepared of these fragments and is sequenced from both ends on an ultra-high throughput sequencing platform, generating a binary contact profile that contains millions of potential interactions.

Another embodiment of the present invention is an improved method for determination of the structural organization of chromatin having less noise and higher resolution. The improved method comprises providing chromatin having DNA cross-linked to protein such that the structural organization of the chromatin is preserved, producing cross-linked protein:DNA complexes by cutting the chromatin with a restriction enzyme; substantially immobilizing the cross-linked protein:DNA complexes on a surface and removing non-crosslinked DNA generated by digesting the chromatin, ligating the cross-linked protein:DNA complexes intramolecularly and removing DNA molecules without a ligation junction, preferably by washing. Preferably, the chromatin is digested with a restriction enzyme that produces a 5′ overhang of at least two non-identical bases. Preferably, the method includes sequencing the DNA of the ligated protein:DNA complexes.

The immobilizing the cross-linked protein:DNA complexes reduces the frequency of intermolecular ligations during the ligation. Preferably, the protein:DNA complexes are substantially immobilized by tethering the protein:DNA complexes on the surface of one or more media selected from the group consisting of beads, matrix, and gel. Preferably, DNA molecules without a ligation junction are removed by an exonuclease and washing.

Another embodiment of the present invention is a kit for determining the three-dimensional arrangement of chromatin in a cell. The kit comprises a cross-linking reagent for cross-linking the DNA and proteins of the chromatin; a lysing reagent. a restriction enzyme for producing cross-linked protein:DNA complexes;(or any other chemical, physical, or enzymatic method for cutting DNA). a substrate for substantially immobilizing the cross-linked protein:DNA complexes; and one or more ligating reagents (or any other reagents or procedure, such as nick-translation, that can connect two molecules of DNA). The kit preferably contains instructions for preparing a library of protein:DNA complexes consistent with this disclosure.

### BRIEF DESCRIPTION OF THE DRAWINGS

FIG. 1(a) is a diagram depicting the surface immobilization of protein:DNA complexes in accordance with the methods of the present invention. FIG. 1(b) illustrates protein:DNA complexes in the absence^{.}of surface immobilization in solution. When protein:DNA complexes are immobilized on a surface, similar to (a), rotational movements and translational diffusion of complexes relative to each other is limited, therefore intramolecular interactions become more likely. When protein:DNA complexes are not immobilized and are free to diffuse in solution, even at low concentrations, intermolecular interactions are difficult to prevent because the complexes can diffuse and find each other.

FIG. 2 is a graph comparing interchromosomal versus intrachromosomal interaction frequencies in TCC and Hi-C experiments. Shown are the percentage of interchromosomal and intrachromosomal interactions in different conditions with TCC, and Hi-C. The blue columns represent the percentage of intrachromosomal interactions and the red columns are the percentages of intermolecular interactions for each experiment. Higher intrachromosomal interaction percentages, which means lower interchromosomal interactions at the same time, indicate lower intermolecular ligations and therefore lower levels of noise in the experiment. All numbers on the table are percentage points. TCC experiments (HindIII TCC and MboI TCC) have been carried out with surface immobilization incorporated to the protocol. Hi-C experiments are carried out without surface immobilization and in the solution. Those results marked with an “*” represent data tabulated from Erez Lieberman-Aiden et al. Science 326, 289 (2009). The “Random” data is the expected proportions of inter and intrachromosomal interactions assuming 100% intermolecular ligations in human genome given the size and number of the chromosomes. Basically the “Random” column shows what the interchromosomal and intrachromosomal interaction frequencies are expected to be when 100% of the data is noise. The expected average fragment size after complete digestion of human genome with the enzyme of interest is 401 bp for MboI, 3416 bp for HindIII and 3805 bp for NcoI. This data shows that surface immobilization significantly reduces the number of interchromosomal interaction and therefore intermolecular ligations. With MboI digestion, which provides smallest fragment sizes and therefore highest resolution, TCC produces only 35% interchromosomal interactions while Hi-C, which lack surface immobilization, produces 75% interchromosomal interactions. In otherwords, when HindIII is used as the restriction enzyme, surface immobilization in TCC causes a roughly 90% decrease in noise. When MboI is used as the restriction enzyme to obtain higher resolution, surface immobilization in TCC causes a roughly 150% decrease in noise. All data here except for MboI Hi-C and “Random” data is obtained by sequencing more than 5 million individual DNA molecules, therefore no error bars are needed as the length of the error bars tend to 0 in this case. “Random” is measured computationally and is therefore absolute and doesn't need error bars. Only MboI Hi-C has not undergone ultra-high throughput sequencing and is a result of sequencing more than 150 individual DNA molecules. This is because the amount of noise in this experiment is so high that the results are believed to be unsuitable for ultra-high throughput sequencing.

FIG. 3 is an interaction frequency heatmap of human chr11 generated using TCC. The interaction frequency map of chromosome 11 shows the interaction intensity between different parts of chr11. X axis starts from the tip of the small arm of chr11 and ends in the end of the long arm on the right. The y axis is the same chromosome in up to down direction. As expected along the diagonal there is extensive interactions, indicating high likelihood of interaction between regions that are close in the linear sequence. The dark-light pattern that seems to repeat itself in four directions from the middle suggests presence of a rosette structure along the chromosome that converges around the centromeric side of the long arm.

### DETAILED DESCRIPTION

The present invention will now be described in detail by referring to specific embodiments as illustrated in the accompanying figures.

One embodiment of the present invention is directed to a genome-wide conformation capture method of determining the three-dimensional arrangement of chromatin in a cell. The method comprises contacting a cell with a cross-linking reagent to cross-link DNA and protein in the cell such that the structural organization of the chromatin or other protein and DNA complexes is preserved; lysing the cell, producing cross-linked protein:DNA complexes by cutting the chromatin using a chemical, physical or enzymatic method, substantially immobilizing the cross-linked protein:DNA complexes, ligating the cross-linked protein:DNA complexes intramolecularly such that the ligated protein:DNA complexes represent structural organization of the chromatin; characterizing the ligated DNA by sequencing or other methods; and identifying any structural organization of the chromatin. Preferably, the sequencing is massively parallel or ultrahigh-throughput sequencing. The structural organization preferably includes information relating to interacting loci of the chromatin.

Chromatin is the complex combination of DNA and proteins that makes up chromosomes. It is found inside the nuclei of eukaryotic cells. Several major components of chromatin are DNA and proteins including histone proteins, transcription factors, and other chromosomal proteins. Chromatin functions, in part, to package DNA into a smaller volume to fit in the cell and to control expression and DNA replication. Chromatin structure is very complex and includes the formation of nucleosomes, in which DNA wraps around histone proteins. The nucleosomes are themselves folded through a series of successively higher order structures to eventually form a chromosome. This “higher order” structural organization both compacts DNA and creates an added layer of regulatory control which ensures correct gene expression. Given the complex structural arrangement of chromatin in eukaryotic cells, chromatin structures which are thousands or even millions of base pairs distant on a chromosome may nonetheless be spatially close to each other and/or represent interacting locations (or loci) of the chromosome.

The methods of the present invention may generally be used in connection with the study of any eukaryotic cell. The cells may be from a variety of organisms, including mammals, such as humans, and non-mammals, such as D. melonogaster. The cells may be from any tissue and may be either normal cells or cells associated with a disease state, such as cancer cells to tumor cells.

In accordance with the present invention, the proteins and DNA of chromatin of the eukaryotic cells of interest are crosslinked with a crosslinking reagent in order to crosslink DNA and protein in the cell such that the structural organization of the chromatin or other protein:DNA complexes is preserved. The cross-linking reagent selected for use in connection with the present inventions is not particularly limited. In general, any cross-linking reagent or a combination of cross-linking reagents may be used to cross-link the DNA and protein in the cell so long as the structural organization of the chromatin or other protein:DNA complexes is preserved. In a preferred embodiment, the crosslinking reagent is formaldehyde. Other suitable crosslinking reagents that can be used alone or in combination with formaldehyde include but are not limited to DSS (Disuccinimidyl suberate), DSG (Disuccinimidyl glutarate) BASED (Bis-[b-(4-Azidosalicylamido)ethyl]disulfide), DST (Disuccinimidyl tartarate).

In one embodiment, the eukaryotic cells of interest may be crosslinked with a 1% final concentration of formaldehyde at room temperature for an appropriate time. In general, it is expected that crosslinking can be complete after approximately 10 min. If desirable, crosslinking may be stopped by adding an appropriate agent, such Cysteamine Hydrochloride to a final concentration of, for example 50 mM, and after incubation with Cysteamine, glycine may be added to a final concentration of, for example, 125 mM. The resulting cells having crosslinked protein:DNA complexes may collected by centrifugation, washed with, for example, 20 mL Phosphate Buffer Saline solution and re-collected by centrifugation.

In accordance with the present invention, the cells are preferably lysed. Lysis generally refers to the breaking down of a cell, usually by viral, mechanical, detergent, enzymatic or osmotic action mechanisms that compromise cellular integrity. The eukaryotic cells of interest are preferably lysed using a lysis buffer selected as appropriate for the cells of interest. Lysis may be done, for instance, with a buffered solution of Hepes, NaCl, Igepal and 1× protease inhibitors.

Optionally, the crosslinked chromatin may be denatured after crosslinking. Denaturation generally refers to a process in which proteins lose their quaternary, tertiary or secondary structure. However, since the protein:DNA have already been crosslinked, the structural information is maintained.

In accordance with the present invention, the chromatin of the eukaryotic cells of interest are cut using a chemical, physical or enzymatic method in order to produce crosslinked protein:DNA complexes. These protein:DNA complexes are chemical species which have a segment of DNA connected, or bonded to, a protein through a crosslink. More than one segment of DNA may be connected, or bonded to, proteins through a crosslink.

Preferably, the chromatin is cut by restriction digestion. Restriction digestion is an enzymatic technique can be used for cleaving DNA molecules at specific sites (i.e. recognition sites). The cleavage method makes use of an important class of DNA-cleaving enzymes called restriction endonucleases or restriction enzymes, and they are able to cleave DNA molecules at the positions at which particular short sequences of bases are present. The theoretical resolution limit of any genome-wide conformation capture is determined by the size of fragments generated by the restriction enzyme(s) used. Therefore, it is preferred to use a restriction enzyme with a small recognition site, because the smaller the recognition size leads to smaller fragments. The recognition sequences are most commonly 4 or 6 bases long. Preferred restriction enzymes for use in present invention create an overhang of at least two nonidentical base pairs. An overhang is a stretch of unpaired bases in the end of a DNA molecule. Preferably, the restriction enzyme selected in connection with the present invention creates a 5′ overhang. For example, suitable restriction enzymes for use in the present invention include MboI which recognizes the sequence 5′-GATC, HINDIII which recognizes the sequence 5′-AAGCTT, and BsrGl, which recognizes 5′-TGTACA. Most preferably, the restriction enzyme used is MboI. In a preferred embodiment, restriction digestion is carried out using 20 μl 10× NEBuffer2, 1 μl of 1M DTT, 75 μl water and 4 μl water 25 U/μl MboI restriction enzyme.

The overhang left by the restriction enzyme may be blunted by known literature methods such that the DNA strands end in a base pair instead of an overhang of unpaired bases. Preferably, the overhang is blunted in manner such that the blunted DNA of the protein:DNA complexes that confers advantageous properties for high resolution conformation capture. Preferably, the overhangs are filled in with an α-thio-triphosphate containing nucleotide analogue inserted before a biotinylated nucleotide. An appropriate α-thio-triphosphate, such as dGTPαS, forms a thiosulphate bond that confers exonuclease resistance to the DNA of the protein:DNA complexes. As will be described subsequently, the insertion of α-thio-triphosphate containing nucleotide before a biotinylated nucleotide permits separation of ligated DNA from DNA that has not been ligated.

In accordance with the present invention, the cross-linked protein:DNA complexes are substantially immobilized. It is not necessary that the protein:DNA complexes be completely immobilized. Rather, it is sufficient that the translational and/or rotational freedom of the protein:DNA complexes is sufficiently restrained in manner that minimizes intermolecular reactions between protein:DNA complexes. It should be stressed that is not necessary to eliminate all intermolecular reaction. Rather, the goal of substantial immobilization is to substantially reduce intermolecular reactions relative to solution and enable removing reagents from cross-linked protein:DNA between different steps of the reaction. Preferably, the protein:DNA complexes are substantially immobilized such that they cannot diffuse freely through the media, and are therefore less likely to encounter another protein:DNA complex. Preferably, substantial immobilization, especially when there is more than one contact point between the surface and the complex, also limits free rotation of the complexes.

Although not limited to this particular order, surface immobilization is done after restriction digestion and before connection of the ends of the protein:DNA complexes intramolecularly.

Preferably, the protein:DNA complexes are substantially immobilized by tethering the protein:DNA complexes to one or more media. The media may be one or more media selected from the group consisting of beads, chips, colloids, matrices, and gels, and the protein:DNA complexes may be tethered on the surface of or on the inside of the media as appropriate for the media. In a preferred embodiment, the protein:DNA complexes are substantially immobilized by a covalent bond between the side-chains of the amino acids of the proteins of chromatin and a reactive chemical group on the surface of or inside of one or more media selected from the group consisting of beads, chip, colloids, matrix, and gel. In another embodiment, the protein:DNA complexes are substantially immobilized by a covalent or non-covalent bond between the DNA of chromatin and a reactive chemical group on the surface or inside of one or more media selected from the group consisting of beads, chip, colloids, matrix, and gel.

Preferably, the protein:DNA complexes are tethered to the surface of the media in a manner designed to minimize interactions between different protein:DNA complexes on the surface of the media. That is to minimize the likelihood of two cross-linked protein:DNA complexes being immobilized next to each other at a distance less than the maximum permissive distance for ligation to take place between them. It is preferred to use media, such as beads, colloids and matrices with a well defined surface area or loading potential to enable appropriate loading of the media with protein:DNA complexes. Preferably, any unused site on the surface of the media is blocked after immobilization of the protein:DNA complexes on the media.

For example, in one embodiment of the present invention, the protein:DNA complexes are substantially immobilized by first biotinylating the proteins of the protein:DNA complexes and then tethering the biotinylated protein:DNA complexes to an appropriate surface. Biotinylation refers generally to a process of covalently attaching a biotin tag to a molecule or surface. The proteins of the protein:DNA complexes may be biotinylated by known methods with Iodoacetyl-PEG₂-Biotin to biotinylate the cysteine residues of all proteins.

The iodoacetyl group of Iodoacetyl-PEG₂-Biotin reacts with reduced thiols (such as the sulfhydryl groups, —SH, on cysteine) at alkaline pH to form stable thioether bond. Thus, in a further embodiment of the present invention, additional thiol groups are added to the proteins of chromatin through a chemical reagent. The thiol groups may added to the proteins of chromatin by reacting the proteins of chromatin with an aminothiol and a crosslinking reagent according to known methods. The thiol groups may be added to the lysines of the proteins of chromatin by reacting them with a cross-linking reagent and cysteamine, for instance, by reacting them with formaldehyde and cysteamine. This may be done during the step where the cells are being cross-linked with formaldehyde.

It is not necessary to biotinlyate the thiol residues. In another embodiment of the present invention, the protein:DNA complexes may be substantially immobilized by biotinylating the N-termini and lysine residues of the proteins of the chromatin. Botinylation reagents containing N-hydroxysuccinimide (NHS) may be used to label proteins at primary amino groups (—NH2), which exist in the side chain of lysine residues and at the N-terminus of each polypeptide.

The biotinlylated protein:DNA complexes may be substantially immobilized by tethering the biotinylated protein DNA complexes to a biotin binding surface. Suitable commercially available biotin binding surfaces include Streptavidin biotin-binding protein coated on to beads or chips, Avidin biotin-binding protein coated on to beads or chips, and NeutrAvidin biotin-binding protein coated on to beads or chips. Streptavidin, Avidin, and Neutravidin are proteins that bind the biotin molecule very strongly and specifically in a way resembling the binding of a ligand to a receptor. Most preferably, Streptavidin biotin-binding protein immobilized to magnetic beads is used to substantially immobilize biotinylated protein:DNA complexes. The magnetic beads provide an way to separate biotinlyated protein:DNA complexes from solutions or other media.

For example, suitable streptavidin beads include DynaBeads MyOne Streptavidin T1 Magnetic Beads (Cat. No. 656-01, Invitrogen, Carlsbad, Calif.). These may be prepared by first washing the beads with PBS mixed with 0.01% Tween 20 (TPBS). In a preferred embodiment, the biotinylated protein:DNA complexes, together with 1 μl 10% Tween 20 and 2 μl 0.5M EDTA are combined with the streptavidin beads followed by rocking at room temperature to immobilize the crosslinked and biotinylated DNA-protein complexes on the surface of the beads. To block free streptavidin, free L-Biotin may added followed by rocking at room temperature. During the washing and reaction, the streptavidin beads may be collected using a magnet.

In accordance with the present invention, the cross-linked protein:DNA complexes are connected intramolecularly such that the connected protein:DNA complexes represent structural organization of the chromatin. In a preferred embodiment, the cross-linked protein:DNA complexes are preferably ligated such that the ligated protein:DNA complexes represent structural organization of the chromatin. DNA Ligation, or DNA ligating, generally refers to the process of joining two pieces of DNA to a single piece, generally through the use of DNA ligase. In the present invention, the intramolecular ligations represent the joining of two pieces of DNA having a spatial affinity to each other, which may be termed interacting loci of the DNA. In a preferred embodiment, these interacting loci are characterized by presence of biotin near the position where the DNA strands have been ligated. In a preferred embodiment, protein:DNA complexes are ligated with a solution of DNA Ligase Buffer and T4 DNA Ligase in water at room temperature.

Other ways of connecting DNA intramolecularly such that the connected protein:DNA complexes represent structural organization of the chromatin may be used. For instance, the DNA may be connected intramolecularly using nick translation as disclosed in U.S. application Ser. No. 12/537,138.

In a preferred embodiment, after ligation, the biotinylated nucleotides on DNA ends that have not participated in ligation are preferably removed. As noted, in one embodiment, blunting of the 5′ overhang may be done preferably with both a biotinylated nucleotide and a nuclease resistant nucleotide. Preferably, the nuclease resistant nucleotide is added before the biotinylated nucleotide. In accordance with this embodiment, biotinylated DNA ends that have not undergone ligation may be removed using an exonuclease. For instance, one suitable exonuclease for use in accordance with the present invention is E. coli Exonuclease III (ExoIII). ExoIII catalyzes removal of mononucleotides from 3′-hydroxyl termini of duplex DNA until it encounters the exonuclease resistant phosphorothioate bond, which is inserted on the 5′ of the biotinylated nucleotide by incorporation of the α-thio-triphosphate containing nucleotide. This treatment removes biotinylated nucleotides from the DNA ends that have not been ligated, but does not completely degrade DNA fragments because “upstream” of the biotin the DNA is protected with a phosphorothioate bond inserted through the nuclease resistance nucleotide such as dATPαS during blunting.

Preferably, after ligation is complete, the crosslinks are removed and the DNA is separated from the protein according to known methods in order to prepare the DNA for sequencing. This may be done by known method, preferably by the use of a suitable proteinase. In a preferred embodiment, proteinase K (NEB, Ipswich, Mass.) is contacted with the surface immobilized protein:DNA complexes and the resulting solution is extracted with Phenol:Chloroform:Isoamylalcohol (25:24:1) and then chloroform. The DNA may then precipitated with NaCl, the addition ice cold 100% ethanol and incubation at −20° C. for an appropriate time. The DNA may be collected by known methods, including centrifugation.

In order to obtain the structural information in the form of at least the interacting loci of the chromatin that was preserved during ligation, it is preferable that the DNA be sheared according to known methods. In one embodiment, the DNA is sheared with a Covaris S2 Instrument (Applied Biosystems, Carlsbad, Calif.) at the following settings: Duty Cycle 5%, Intensity 5, Cycles/Burst 200, and total time 180 seconds. the DNAsample is preferably purified and precipitated by known methods.

As has been noted, in accordance with a preferred embodiment of the invention, the interacting loci of the chromatin are characterized by a biotin junction. The presence of the biotin junction makes it possible to separate out any DNA fragments that do not have a biotin junction, and thus, is not an interacting loci. This is preferably done by binding the biotin junction containing fragment to a biotin binding surface. In a preferred embodiment, streptavidin beads are used to separate the biotin-junction-containing DNA fragments from the non-biotin-junction containing DNA fragments.

The resulting DNA fragments represent a “library” of the interacting loci of the chromatin of the cell. In a preferred embodiment, the DNA fragments are sequenced using known methods and techniques, preferably a parallel sequencing technique. In order sequence the resulting library, it may be necessary to further prepare the library post-shearing. For instance, it may be necessary to blunt the DNA fragments after shearing or to add A overhangs as necessary for PCR amplification or sequencing as is well known in the art.

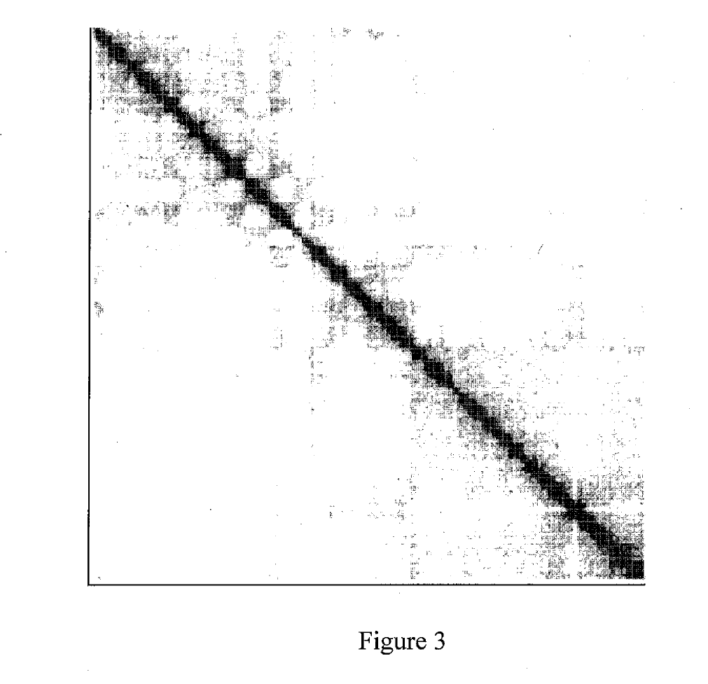

To analyze the data, the human genome may be divided into regions (loci), each comprised of consecutive restriction cut sites, wherein X is the total number of cut sites for the restriction enzyme. A genome-wide contact matrix was defined as a X by X matrix in which each entry for matrix (Y_{i,j)} is the number of times locus i is seen with locus j in the dataset. As noted before (ref, science paper), this interaction matrix, which reflects a two dimensional impression of all contacts that are present in the dataset, can be represented as a heatmap, with the intensity of each pixel corresponding to the contact frequency of the two corresponding loci. See, For instance, FIG. 3.

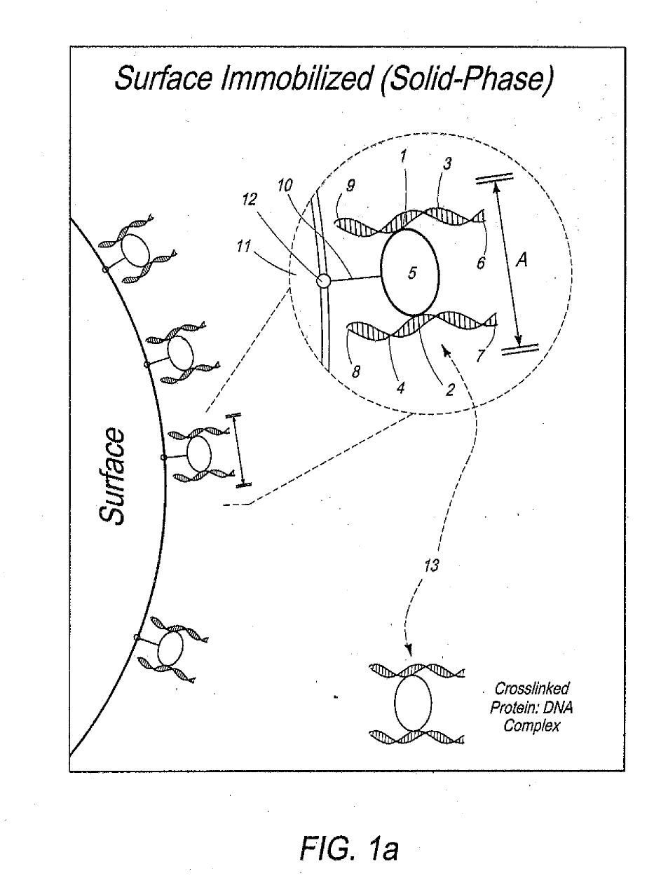

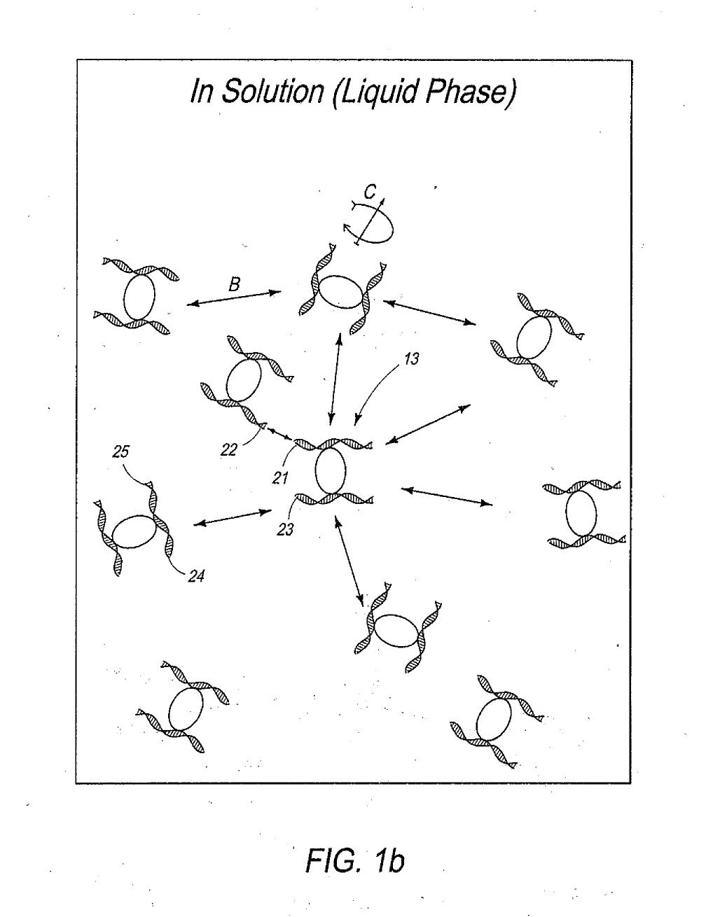

Certain aspects of one embodiment of the present invention may be understood with respect to FIG. 1. In FIG. 1(a), DNA strands 3, 4 of the chromatin are both cross-linked to protein complex 5 of chromatin. The structural relationship between the DNA strands 3, 4 and the protein 5 are maintained in the present invention by the formation of crosslinks 1, 2 during crosslinking. The DNA strands 3, 4 are subsequently cut, by for instance, a restriction enzyme to produce a crosslinked protein:DNA complexe 13 having DNA ends 6, 7, 8, 9. In a preferred embodiment, a restriction enzyme is used such that DNA ends 6, 7, 8, and 9 are characterized by a 5′ overhang, which is subsequently blunted by an exonuclease resistant nucleotide and a biotinylated nucleotide.

The crosslinked protein:DNA complex may then be substantially immobilized on the surface of a media 11 by tethering the protein:DNA complex 13 to media 11 at junction 12. In the embodiment of the FIG. 1(a), the structure 10 which joins the protein to the surface may be, for instance, a biotinylated portion of protein 5 which is attached at junction 12 by a medium having a biotin binding surface. Tethering the protein:DNA complex 13 to the surface of media is believed to substantially reduce its translational motion in at least the direction A shown in FIG. 1(a).

The crosslinked protein:DNA complexes are then connected intramolecularly by joining DNA end 6 to DNA end 7 and/or joining DNA end 9 to DNA end 8. This may be done, for instance, by DNA ligation using DNA ligase. The effect of joining the DNA ends in this manner is to produce protein:DNA complexes in which the structural affinity of DNA strands 3 and 4 is preserved. Preferably, the crosslinks are undone, and the DNA is purified and DNA strands that did not ligate to any other DNA strand at either end are removed. The structural relationship between DNA strands 3 and 4, which is the fact that they were in spatial proximity such that they were crosslinked to each other through a protein complex (in this case protein complex 5), is then revealed by, for instance, sequencing the DNA strands.

In contrast, FIG. 1(b) shows the situation in which protein:DNA complexes 13 are not tethered to the surface of a media and are dissolve in solution. In solution, even dilute solutions, protein:DNA complexes retain substantial translational B and Rotational C degrees of freedom. These translation and rotational degrees of freedom permit, during connection the DNA ends of different protein:DNA complex to intermolecularly ligate. For instance DNA ends 21, 23 of one crosslinked protein:DNA complex may become connected to the DNA ends of a different protein:DNA complex 20, 24, 25 resulting in intermolecular ligation. During sequencing these intermolecularly connected species, which do not contain structural information of the chromatin, read as “noise” because it is not possible to identify a sequence from intermolecular ligation from intramolecular ligation.

### Example 1

Overview. In one example of the present invention, cells were crosslinked with formaldehyde and treated with Iodoacetyl-PEG₂-Biotin to biotinylate the cysteine residues of all proteins. Chromatin was then digested with a restriction enzyme, leaving 5′ overhangs. After digestion, crosslinked protein-DNA complexes were immobilized on the surface of streptavidin-coated magnetic beads through biotinylated proteins and excess streptavidin was blocked. The 5′ overhangs were filled in with an α-thio-triphosphate containing nucleotide analogue inserted before a biotinylated nucleotide. Blunt DNA ends were then ligated while immobilization prevented free diffusion of the complexes, therefore promoting intramolecular ligations. After ligation, DNA is purified, which separates it from the surface and crosslinked proteins. The biotinylated nucleotides on DNA ends that did not participated in ligation were then removed using E. coli Exonuclease III (ExoIII). ExoIII was used to catalyze removal of mononucleotides from 3′-hydroxyl termini of duplex DNA until it encounters the exonuclease resistant phosphorothioate bond, which is inserted on the 5′ of the biotinylated nucleotide by incorporation of the α-thio-triphosphate containing nucleotide. After exonuclease treatment, the DNA was sheared and the fragments that include a ligation junction and had therefore maintained biotin, were isolated on streptavidin-coated magnetic beads. A library was prepared of these fragments and sequenced from both ends on an ultra-high throughput sequencing platform, generating a binary contact profile that contains millions of potential interactions.

Initial Preparation of the Cells. 25 million GM12878 human lymphoblast cells (Coriell, Camden, N.J.) were crosslinked with 1% final concentration of formaldehyde at room temperature for 10 min. Crosslinking was stopped by adding Cysteamine Hydrochloride (Sigma-Aldrich, St Louis, Mo.) to a final concentration of 50 mM for 5 min. After incubation with Cysteamine, glycine was added to a final concentration of 125 mM followed by incubation at room temperature for 5 minutes and on ice for 15 min. Cells were centrifuged at 4° C. with 700 g for 10 min. The supernatant was discarded.

The resulting pellet was re-suspended in 20 mL Phosphate Buffer Saline solution (PBS). The cells were spun down at 4° C. with 700 g for 10 min. The steps of re-suspending the pellets and spinning them down were repeated twice.

Cell Lysis and Protein Biotinylation. The cell pellet was re-suspended in 550 μl of ice cold Lysis Buffer and incubated on ice for 15 min. The lysis buffer contains 10 mM Hepes pH=8.0, 10 mM NaCl, 0.2% Igepal CA630, and 1× protease inhibitors (Roche Ltd, Basel, Switzerland). The cells were transferred to an ice cold Dounce homogenizer (Wheaton Industries Inc. Millville, N.J.) and treated with 10 strokes of pestle A, one minute incubation on ice, and 10 more strokes of pestle A.

The nuclei were pelleted by centrifugation at 4° C. and 5000 g for 5 min. The supernatant was discarded, and the pellet was washed with 50 mM Hepes pH=8.0, 50 mM NaCl, 1 mM EDTA. The steps of pelleting the nuclei, discarding the supernated and washing the pellet were repeated.

The resulting pellet was re-suspended in 250 μl 50 mM Hepes pH=8.0, 50 mM NaCl, 1 mM EDTA and aliquot into 5 tubes. 19 μl of 2% Sodium Dodecyl Sulfate (SDS) was added per tube followed by incubation at 60° C. for 15 minutes to denature the chromatin.

The tubes were cooled down to room temperature, and EZLink-Iodoacetyl-PEG2-Biotin (IPB, 21 μl 25 mM) (Piece Protein Research Products, ThermoScientifc, Rockford, Ill.) was added to each tube followed by rocking at RT and in dark for 60 min to biotinylate all proteins of the lysate. 260 μl 1× NEBuffer2 (New England Biolabs, Ipswich, Mass.) was added to each tube. The tubes were put on ice and 45 μl 10% Triton X-100 was added to each tube and mixes followed by incubation on ice for 15 min to neutralize SDS.

Digestion of the Chromatin. 20 μl 10× NEBuffer2, 1 μl of 1M DTT, 75 μl water and 4 μl 25 U/μl MboI restriction enzyme (New England Biolabs, Ipswich, Mass.) were added to each tube. MboI cuts the sequence 5′-GATC before G and after C on the top and the bottom strands, respectively, leaving a 5′ overhang. Incubation was performed on a shaking incubator at 37° C. overnight while shaking at 275 rpm.

Elimination of Excess IPB. To eliminate excess IPB the 5 sample tubes were mixed and dialyzed against 2 liters of 10 mM Tris.HCl pH=8.0, 1 mM EDTA for 5 hours at room temperature. A 20 kD cutoff dialysis cassette (Piece Protein Research Products, ThermoScientifc, Rockford, Ill.) was used. The buffer was renewed after 2 hours. The dialyzed mixture, which contains chromatin DNA was transferred to another tube.

Surface Immobilization. In this procedure, during the various washing and reaction steps the beads were collected on the wall of the tube using a magnet. The solution was then aspirated out of the tube. After the tube was removed from the magnet, the beads were re-suspended in the appropriate buffer. All washing steps were done with 500 μl of the buffer unless otherwise indicated.

400 μl DynaBeads MyOne Streptavidin T1 Magnetic Beads (Cat. No. 656-01, Invitrogen, Carlsbad, Calif.) were washed three times with PBS mixed with 0.01% Tween 20 (TPBS). MyOne beads have a surface area of approximately 250 cm²/ml and therefore the amount used is roughly 100 cm² surface area to immobilize the digested chromatin. The Beads were resuspended in 2 mL TPBS.

The dialyzed chromatin mixture was split in 5 tubes equally and 0.5 μl 10% Tween 20 and 2 μl 0.5M EDTA were added to each tube. 400 μl DynaBeads in TPBS mixture that had been prepared was added to each tube followed by rocking at room temperature for 30 minutes to immobilize the crosslinked DNA-protein complexes (chromatin) on the surface of the beads. To block free streptavidin, 25 μl 5 mM free L-Biotin was added to each tube followed by rocking at room temperature for 15 minutes.

The beads were washed once with TPBS. The beads were washed once with 10 mM Tris.HCl pH=8.0, 50 mM NaCl, 0.4% Triton X-100 and resuspended in 100 μl of the same buffer.

Blunting the 5′ Overhangs. 59 μl water, 1 μl MgCl₂, 10 μl 10× NEBuffer2, 0.7 μl 10 mM dGTP, 0.7 μl 10 mM dTTP, 0.8 μl 10 mM dATPαS (2′-Deoxyadenosine-5′-O-(1-thiotriphosphate) sodium salt, Sp-isomer, Cat. No. BLG-D007-05, AXXORA, LLC, San Diego, Calif.), 15 μl 0.4 mM Biotin-14-dCTP (Invitrogen, Carlsbad, Calif.), 4 μl 10% Triton X-100 and 5 μl 5 u/μl klenow DNA polymerase (M0210S, NEB, Ipswich, Mass.) were added to each tube to start blunting. The tubes were rocked at RT for 40 minutes. The beads were then washed twice with 50 mM Tris.HCl pH=7.4, 0.4% Triton X-100, 0.1 mM EDTA and re-suspended in 500 μl of the same buffer. The contents of each of the five tubes were transferred to a 15 ml conical tube.

Intramolecular Ligation. 4000 μl water, 500 μl 10×T4 DNA Ligase Buffer (NEB, Ipswich, Mass.), 90 μl 20% Triton X-100 and 2 μl 2000 U/μl T4 DNA Ligase (M0202M, NEB, Ipswich, Mass.) were added to each tube to start blunt end ligation. The tubes were rocked at 16° C. for 4 hours. The supernatant was discarded and beads were re-suspended in 80 μl 25 mM Tris.HCl pH=8.8, 0.2% SDS, 2 mM EDTA, 125 mM NaCl per tube and combined into one tube for a total volume of 400 μl.

Reversing the Crosslinks and DNA Extraction. 30 μl 20 mg/ml proteinase K (NEB, Ipswich, Mass.) was added to the tube. The tube was incubated at +65 degree Celsius overnight. The beads were collected to the wall of the tube with a magnet and the supernatant was transferred to a new tube. The beads were discarded. The supernatant was extracted three times with Phenol:Chloroform:Isoamylalcohol (25:24:1) and once with chloroform with standard protocol using Heavy Phase Lock Tubes (Fisher Scientific Catalogue No.: FP2302830). The DNA was then precipitated with 18 μl 5M NaCl and addition of 850 μl of ice cold 100% EtOH. The tube was incubated at −20° C. for 1 hour to precipitate DNA.

DNA was pelleted with centrifugation at 20,000 g at 4° C. for 10 min. The supernatant was discarded and pellet was washed with 80% EtOH. The pellet was air-dried and dissolved in 50 μl water.

Removal of Biotin From non-ligated DNA ends. 50 μl of the extracted DNA was treated with 3 μl 100 U/μl ExoIII (M0206S, NEB, Ipswich, Mass.) in a total volume of 90 μl NEBuffer1 for 1 hour at 37° C. This treatment removes biotinylated nucleotides from the DNA ends that have not been ligated, but won't completely degrade DNA fragments because upstream of the biotin the DNA is protected with a phosphorothioate bond inserted through dATPαS during blunting. The reaction was stopped with 2 μl 0.5M EDTA and 2 μl 5MNaCl. The enzyme was heat-inactivated with incubation at 70° C. for 20 minutes.

DNA Fragmentation (Covaris). A Covaris S2 Instrument (Applied Biosystems, Carlsbad, Calif.) was set to 4° C. and degassed for 30 minutes. The sample was transferred to a 6 by 16 mm AFA fiber microtube with snap-cap recommended for Covaris. DNA shearing was done with the following settings: Duty Cycle 5%, Intensity 5, Cycles/Burst 200, and total time 180 seconds. After shearing, sample was transferred to another tube and purified with one round of Phenol:Chlorofom:Isoamylalcohol (25:24:1) and once with chloroform with standard protocol using Heavy Phase Lock Tubes (Fisher Scientific Catalogue No.: FP2302830). The DNA was then precipitated with 18 μl 5M NaCl and addition of 850 μl of ice cold 100% EtOH. The tube was incubated at −20° C. for 1 hour to precipitate DNA.

The DNA was pelleted with centrifugation at 20,000 g at 4° C. for 10 minutes. The supernatant was discarded and pellet was washed with 80% EtOH. The pellet was air-dried and dissolved in 75 μl water.

End-Repair. The following reagents were added to the sample tube (75 μl) to start end-repair: 10 μl T4 DNA Ligase Buffer (NEB, Ipswich, Mass.), 4 μl 10 mM dNTPs mix, 5 μl 10 U/μl T4 PNK, 1 μl 5 U/μl klenow (NEB, Ipswich, Mass.) and 5 μl 3 U/μl T4 DNA Polymerase (NEB, Ipswich, Mass.). The tube was incubated at 20° C. for 30 minutes after which DNA was purified with QiaQuick PCR purification kit (Qiagen, Valencia, Calif.) into 45 μl Elution Buffer per manufacturer instructions.

Adding A-Overhangs. To add the A-overhang to all DNA ends, the following reagents were added to the purified sample in Elution Buffer: 5 μl 10× NEBuffer2, 1 μl 10 mM dATP, and 3 μl 5 U/μl exo-klenow fragment (NEB, Ipswich, Mass.) followed by incubation at 37° C. for 30 minutes.

Removal of all Non-Ligation-Junction-Containing DNA from the Library. This step results in removal of all non-ligation-junction-containing DNA fragments from the library. It will also produce a library compatible with massively-parallel sequencing on a Genome Analyzer Platform (Illumina Inc. San Diego, Calif.). In this step, during various washing and reaction steps the beads were collected on the wall of the tube using a magnet. The solution was then aspirated out of the tube. After tube was removed from the magnet the solution was re-suspended in the appropriate buffer. All washing steps were done with 500 μl of the buffer unless otherwise indicated.

10 μl DynaBeads MyOne Strepavidin T1 were washed twice with 1× Bind&Wash buffer (B&W) in low-bind tubes, per manufacturer instructions. The beads were re-suspended in 50 μl 2×B&W and mixed with the sample (A-overhang adding reaction) followed by rocking at RT for 30 minutes. The beads were separated with a magnet and supernatant was removed. The beads were then re-suspended in 50 μl Quick Ligation Reaction Buffer (M2200S, NEB, Ipswich, Mass.).

The following were mixed with the beads to ligate Genome Analyzer adaptors to all DNA ends: 40 μl water, 6 μl PE Adaptors (Illumina Inc) and 5 μl Quick T4 DNA Ligase (M2200S, NEB, Ipswich, Mass.). This was followed by rocking at room temperature for 20 minutes.

The ligation mixture was discarded and beads were washed three times with 10 mM Tris Buffer to completely removed unbound DNA fragments, and the beads were re-suspended in 10 μl water.

PCR Amplification, Size Selection and Production of Final Library. PCR was carried out with Illumina Genome Analyzer PE PCR primers per manufacturer's protocol with 14 cycles using all the bead mixture as template in the PCR reaction. The PCR product was run on a 2% agarose gel and the bands between 300 bp and 500 bp were extracted from the gel using Qiagen Gel Extraction Kit (Qiagen, Valencia, Calif.) and eluted into 30 μl EB. This is the final library and can be sequenced after quantification.

Massively Parallel Sequencing of Final Library. Sequencing was carried out on a Genome Analyzer IIx Instrument (Illumina Inc, San Diego, Calif.) per manufacturer's instruction.

### Example 2

We created a TCC library from 25 million GM12878 human lymphoblast cells (Coriell Institute, Camden, N.J.) using HindIII as the restriction enzyme. For comparison, a Hi-C library of 25 million GM12878 cells digested with HindIII was also created as described by Lieberman et al. The two libraries were sequenced on Illumina Genome Analyzer II platform (Illumina, San Diego, Calif.) in a paired-end format. For every dataset, the two reads of each cluster were filtered for ligation junctions and then aligned to build GRCh37/hg19 of the human genome using Bowtie (Langmead et al., 2009).

Two types of read pairs do not contain information about the 3D organization of the genome and are a result of error in the process. The first group is the DNA molecules that do not include a ligation junction yet bind to streptavidin coated beads and appear in the final library. In sequencing, these molecules which we refer to as “dirt” result in read pairs that align only 300-700 bp apart to opposite strands of the genome. The second group is the DNA molecules that ligate to themselves during intramolecular ligation, forming a circular DNA. In sequencing, such molecules result in read pairs that align close to one another on opposite strands of template. The distance between such pairs cannot be more than the size of the largest fragment generated by the restriction enzyme used. To filter such pairs from the datasets, we removed all pairs that aligned on opposite closer than 30,000 bp when HindIII was used, and closer than 12,000 bp when MboI was used. All but one copy of pairs that were suspected of being a result of PCR duplication were also removed from the datasets.

Thus, after alignment, all the pairs that were potentially from DNA fragments without any ligation junction or a result of ligation of the ends of a single molecule to each other were removed from the dataset.

To analyze the data, the human genome was divided into 3004 regions (loci), each comprised of 277 consecutive HindIII cut sites. A genome-wide contact matrix was defined as a 3004 by 3004 matrix (M277) in which the entry for m277_{i,j} is the number of times locus i is seen with locus j in the dataset. This interaction matrix, which reflects a two dimensional impression of all contacts that are present in the dataset, can be represented as a heatmap, with the intensity of each pixel corresponding to the contact frequency of the two corresponding loci. See FIG. 3. Such heatmaps obtained from cells of the same tissue at different stages or cells of different tissues can be used to compare the three dimensional organization of chromatin in those cells. For example, one can compare the three dimensional organization of cancer cells to regular cells from the same tissue, or to tissues that are suspected of tumorigenic potential for diagnostic purposes. One can also compare the three dimensional organization of chromatin in a certain cancer tumor to different subtypes of the same cancer or different cancers to identify the most effective course of treatment.

### Example 3 (TCC has Reduced Level of Noise)

Differing intermolecular ligation levels should manifest itself in the portion of interchromosomal interactions in the data. In a random ligation of HindIII-digested human DNA, 95% of all ligation events are expected to take place between fragments that belong to different chromosomes. In other words, in a Hi-C or a TCC experiment, 95% of the noise caused by intermolecular ligations comes in the form of interchromosomal connections. These random ligations are expected to be uniformly distributed throughout the interaction matrix.

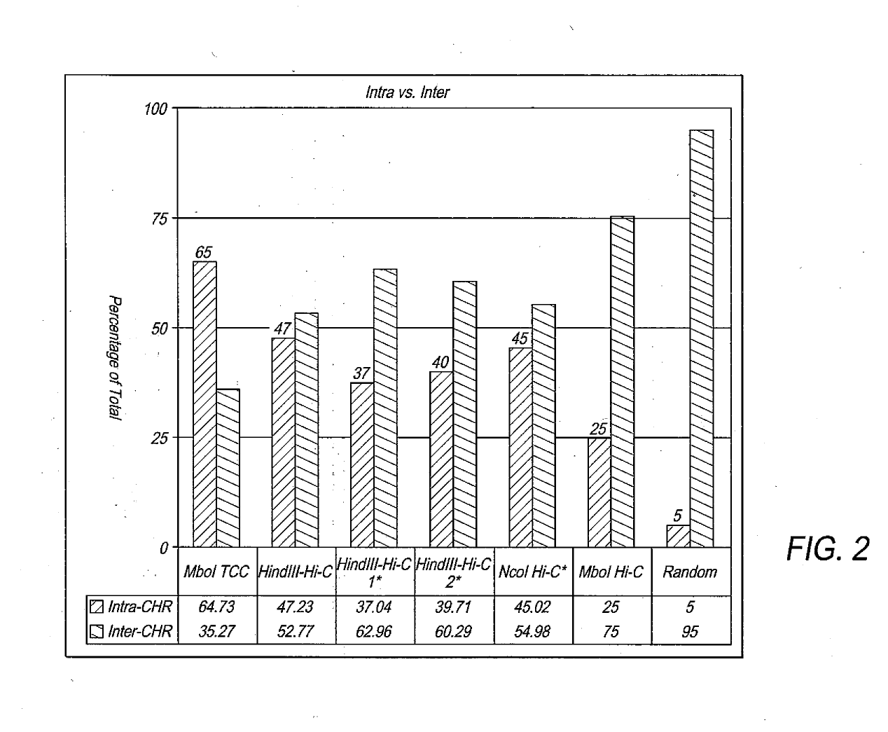

Due to existence of chromosome territories (Cremer and Cremer, 2001) and polymer-like properties of DNA (Lieberman-Aiden et al., 2009), a higher portion of contacts than expected by random that in fact are present in the cells are expected to be intrachromosomal. This is why intermolecular ligation noise appears mostly as interchromosomal interactions in the data. The portion of intrachromosomal and interchromosomal contacts in each dataset was compared. We observe that the percentage of intrachromosomal contacts in TCC is significantly higher than that of Hi-C (FIG. 2). This strongly supports lower levels of intermolecular ligation noise in TCC (FIG. 2). In fact, for the first time, these TCC results reveal the dominance of chromosome territories; an absolute majority of all interactions inside the nucleus take place intrachromosomally.

### Example 4

The theoretical resolution limit of a genome-wide conformation capture assay such as Hi-C or TCC is determined by the size of fragments generated by the restriction enzyme(s) used. To increase resolution limit, we used MboI, a 4-cutter restriction enzyme. MboI is expected to produce fragments of 401 bp average size upon complete digestion of the human genome, this is while HindIII is a 6-cutter restriction enzyme that is expected to produce fragments of 3416 bp average size upon complete digestion. This is expected to translate to a roughly 9 time decrease in fragment sizes and theoretical resolution limit in a Hi-C or TCC experiment. This increase in the resolution limit brought about by MboI comes with an equivalent increase in concentration of DNA ends in the reaction, which makes preventing intermolecular ligations more difficult.

A Hi-C library of 25 million GM12878 cells using MboI as restriction enzyme was prepared. Characterizing 164 individual DNA molecules from this library shows that 75% of all connections in this library are interchromosomal and only 25% intrachromosomal (FIG. 2). These values suggest rampant intermolecular ligation noise, which renders the library unsuitable for ultra-high throughput sequencing.

A TCC library of 25 million GM12878 cells using MboI was prepared. This MboI-TCC library was sequenced on IIlumina Genome Analyzer II platform (Illumina, San Diego, Calif.). Analysis of all eligible connections in this library shows that only 35% of all connections are interchromosomal while the rest are intrachromosomal (FIG. 2). Comparison of MboI-TCC data to MboI-Hi-C data underscores the superior performance of surface-immobilized conformation capture in conditions of equal total DNA amounts but higher concentrations of DNA ends and its effectiveness in preventing intermolecular ligations. Also, the fact that MboI-TCC and Hi-C perform equally well demonstrates consistency of surface-immobilized conformation capture despite dramatic change in the conditions of the experiment. This is in stark contrast to Hi-C, which shows widely varying levels of intermolecular ligation noise even in the replicates of identical conditions (Lieberman-Aiden et al., 2009). These results also show that surface-immobilization makes higher resolution analysis in genome-wide conformation capture assays possible.

### Other Embodiments

Although the present invention has been described in terms of specific exemplary embodiments and examples, it will be appreciated that the embodiments disclosed herein are for illustrative purposes only and various modifications and alterations might be made by those skilled in the art without departing from the spirit and scope of the invention as set forth in the following claims.

### REFERENCES

The following references are incorporated herein in their entirety:
Cremer, T., Cremer, M., Dietzel, S., Muller, S., Solovei, I., and Fakan, S. (2006). Chromosome territories—a functional nuclear landscape. Current opinion in cell biology 18, 307-316.
Dekker, J., Rippe, K., Dekker, M., and Kleckner, N. (2002). Capturing chromosome conformation. Science (New York, N.Y. 295, 1306-1311.
Dostie, J., Richmond, T. A., Arnaout, R. A., Selzer, R. R., Lee, W. L., Honan, T. A., Rubio, E. D., Krumm, A., Lamb, J., Nusbaum, C., et al. (2006). Chromosome Conformation Capture Carbon Copy (5C): a massively parallel solution for mapping interactions between genomic elements. Genome research 16, 1299-1309.
Wolffe, A. (1998). Chromatin: Structure and Function. Academic Press San Diego, Calif.

Cremer T., Cremer C. (2001). Chromosome territories, nuclear architecture and gene regulation in mammalian cells. Nat Rev Genet 4, 292-301.

Osborne, C. S., Chakalova, L., Brown, K. E., Carter, D., Horton, A., Debrand, E., Goyenechea, B., Mitchell, J. A., et al. (2004). Active genes dynamically colocalize to shared sites of ongoing transcription. Nature genetics 36, 1065-1071.

Lee, G. R., Spilianakis, C. G., and Flavell, R. A. (2005). Hypersensitive site 7 of the TH2 locus control region is essential for expressing TH2 cytokine genes and for long-range intrachromosomal interactions. Nature immunology 6, 42-48.

Spilianakis, C. G., and Flavell, R. A. (2004). Long-range intrachromosomal interactions in the T helper type 2 cytokine locus. Nature immunology 5, 1017-1027.

Cai, S., Lee, C. C., and Kohwi-Shigematsu, T. (2006). SATB1 packages densely looped, transcriptionally active chromatin for coordinated expression of cytokine genes. Nature genetics 38, 1278-1288.

Simonis, M., Klous, P., Splinter, E., Moshkin, Y., Willemsen, R., de Wit, E., van Steensel, B., and de Laat, W. (2006). Nuclear organization of active and inactive chromatin domains uncovered by chromosome conformation capture-on-chip (4C). Nature genetics 38, 1348-1354.

Zhao, Z., Tavoosidana, G., Sjolinder, M., Gondor, A., Mariano, P., Wang, S., Kanduri, C., Lezcano, M., Sandhu, K. S.,

Lieberman-Aiden E, van Berkum N L, Williams L, Imakaev M, Ragoczy T, Telling A, Amit I, Lajoie B R, Sabo P J, Dorschner M O, Sandstrom R, Bernstein B, Bender M A, Groudine M, Gnirke A, Stamatoyannopoulos J, Mirny L A, Lander E S, Dekker J. (2009)

What is claimed is:

1. A method of determining the three-dimensional arrangement of chromatin in a cell, comprising:
Contacting a cell with a cross-linking reagent to cross-link DNA and protein in the cell such that the structural organization of the chromatin or other protein:DNA complexes is preserved;
lysing the cell;
producing cross-linked protein:DNA complexes by cutting the chromatin using a chemical, physical or enzymatic method;
substantially immobilizing the cross-linked protein:DNA complexes;
connecting the cross-linked protein:DNA complexes intramolecularly such that the connected protein:DNA complexes represent structural organization of the chromatin;
characterizing the DNA of the protein:DNA complexes by sequencing or other methods; and
identifying any structural organization of the chromatin.

2. The method of claim 1, further comprising, denaturing the chromatin.

3. The method of claim 1, wherein the protein:DNA complexes are cut by restriction digestion;

4. The method of claim 1, wherein the protein:DNA complexes are substantially immobilized by tethering the protein:DNA complexes to one or more media.

5. The method of claim 4, where the media is selected from the group consisting of beads, chip, colloids, matrix, and gel.

6. The method of claim 1, wherein the protein:DNA complexes are substantially immobilized by a covalent or non-covalent (streptavidin-biotin bonding is not covalent but is very strong) bond between the side-chains of the amino acids of the proteins of chromatin and a reactive chemical group on the surface or inside of one or more media selected from the group consisting of beads, chip, colloids, matrix, and gel.

7. The method of claim 1, wherein the protein:DNA complexes are substantially immobilized through modifying the proteins of the chromatin so to anchor the modified protein:DNA complexes to the surface or inside of one or more media selected from the group consisting of beads, colloids, matrix, and gel.

8. The method of claim 1, wherein the protein:DNA complexes are substantially immobilized by biotinylating the proteins of the chromatin so to anchor the biotinylated protein:DNA complexes to a biotin binding surface

9. The method of claim 8, wherein the protein:DNA complexes are substantially immobilized by biotinylating the thiol groups of the proteins of the chromatin so to anchor the biotinylated protein:DNA complexes to a biotin binding surface.

10. The method of claim 8, wherein the protein:DNA complexes are substantially immobilized by biotinylating the cysteine residues of the proteins of the chromatin so to anchor the biotinylated protein:DNA complexes to a biotin binding surface.

11. The method of claim 8, wherein the protein:DNA complexes are substantially immobilized by biotinylating the N-termini and lysine residues of the proteins of the chromatin so to anchor the biotinylated protein:DNA complexes to a biotin binding surface.

12. The method of claim 8, wherein the protein:DNA complexes are substantially immobilized by biotinylating the glutamate or aspartate residues of the proteins of the chromatin so to anchor the biotinylated protein:DNA complexes to a biotin binding surface.

13. The method of claim 9, wherein thiol groups are added to the proteins of chromatin through a chemical reagent.

14. The method of claim 12, wherein thiol groups are added to the proteins of chromatin through reacting the proteins of chromatin with an aminothiol and a crosslinking reagent.

15. The method of claim 14, wherein thiol groups are added to the lysines of the proteins of chromatin by reacting them with a cross-linking reagent and cysteamine.

16. The method of claim 14 wherein thiol groups are added to the lysines of the proteins of chromatin by reacting them with formaldehyde and cysteamine.

17. The method of claim 8, wherein the substrate is streptavidin-coated chips or magnetic beads.

18. The method of claim 1, wherein the cells are denatured with Sodium Dodecyl Sulfate.

19. The method of claim 4, wherein the chromatin is digested with a restriction enzyme that produces a 5′ overhang of at least two non-identical bases.

20. The method of claim 19, wherein the 5′ overhang is blunted.

21. The method of claim 20, wherein the connection of the protein:DNA complexes intramolecularly is done by blunt-ended ligation using DNA ligase.

22. The method of claim 20, wherein blunting is done with nucleotide analogues.

23. The method of claim 20, wherein a biotinylated nucleotide is used for blunting.

24. The method of claim 20, wherein a nuclease resistant nucleotide analogue is used in blunting.

25. The method of claim 20, wherein a 2-deoxy-nucleoside-5′-(alpha-thio)-triphosphate is used in blunting.

26. The method of claim 1, wherein after the connecting step, protein:DNA complexes that have not undergone connection are removed.

27. The method of claim 1, wherein the sequencing is massively parallel or ultrahigh-throughput sequencing.

28. The method of claim 1, wherein the structural organization is interacting loci in the nucleus of the cell.

29. An improved method for determination of the structural organization of chromatin having less noise and higher resolution, said improved method comprising:
providing chromatin having DNA cross-linked to protein such that the structural organization of the chromatin is preserved;
producing cross-linked protein:DNA complexes by cutting the chromatin with a restriction enzyme;
substantially immobilizing the cross-linked protein:DNA complexes on a surface and removing non-crosslinked DNA generated by digesting the chromatin;
connecting the cross-linked protein:DNA complexes intramolecularly and removing DNA molecules without a connection;

30. The method of claim 29, further comprising sequencing the DNA of the connected protein:DNA complexes.

31. The method of claim 29, wherein the chromatin is digested with a restriction enzyme that produces a 5′ overhang of at least two non-identical bases

32. The method of claim 29, wherein the protein:DNA complexes are substantially immobilized by tethering the protein:DNA complexes on the surface of one or more media selected from the group consisting of beads, matrix, and gel. (see claim 3)

33. The method of claim 29, wherein the non-crosslinked DNA generated by digesting the chromatin is removed by washing.

34. The method of claim 29, wherein the immobilizing the cross-linked protein:DNA complexes reduces the frequency of formation of inter-molecular connections.

35. The method of claim 29, wherein DNA molecules without a connection junction are removed by an exonuclease.

36. A kit for determining the three-dimensional arrangement of chromatin in a cell, comprising:
A cross-linking reagent for cross-linking the DNA and proteins of the chromatin;
a lysing reagent;
a denaturing reagent;
a restriction enzyme for producing cross-linked protein:DNA complexes; (or any other chemical, physical, or enzymatic method for cutting DNA),
a substrate for substantially immobilizing the cross-linked protein:DNA complexes; and
one or more connecting reagents for connecting the protein:DNA complexes intramolecularly.

37. The method of claim 1, wherein protein:DNA complexes are substantially immobilized by a covalent or non-covalent bond between the DNA of chromatin and a reactive chemical group on the surface or inside of one or more media selected from the group consisting of beads, chip, colloids, matrix, and gel.

38. The method of claim 1, wherein connection of the DNA of the crosslinked Protein:DNA complexes intramolecularly is done by ligation using DNA ligase.

39. The method of claim 1, wherein the protein:DNA complexes are substantially immobilized relative to each other.
